(19) United States
(12) Patent Application Publication
(10) Pub. No.: US 2018/0325463 A1
(43) Pub. Date: Nov. 15, 2018

(54) SYSTEM AND METHODS FOR EXTUBATION DEVICE UTILIZATION FOLLOWING LIBERATION FROM MECHANICAL VENTILATION

(71) Applicant: CHILDREN'S MEDICAL CENTER CORPORATION, Boston, MA (US)

(72) Inventor: Brian K. WALSH, Boston, MA (US)

(21) Appl. No.: 15/774,825

(22) PCT Filed: Nov. 11, 2016
(86) PCT No.: PCT/US2016/061650

§ 371 Date: May 9, 2018

Related U.S. Application Data
(60) Provisional application No. 62/254,921, filed on Nov. 13, 2015.

(51) Int. Cl. A61B 5/00; A61M 16/04; A61B 5/0205; G16H 20/30

(52) U.S. Cl. CPC A61B 5/7275; A61M 16/04; A61B 5/7246; A61B 5/021; A61B 5/7475; A61B 5/0002; G16H 20/30; A61B 5/0205

(57) ABSTRACT

In one embodiment, a method for determining patient response to an extubation procedure from a mechanical ventilator is disclosed. The method includes receiving one or more physiologic, pulmonary mechanism or oxygenation-related parameters measurements for a predetermined period of time and retrieving a known data set. A first extubation index is calculated with the physiologic, pulmonary mechanism measurements or oxygenation-related parameters and compared with the known data set. An assessment is produced concerning utilization of a device after extubation.

### CROSS-REFERENCE TO RELATED APPLICATION

This application claims the benefit of priority under 35 U.S.C. § 119(e) to U.S. Provisional Application No. 62/254,921, filed Nov. 13, 2015, which is incorporated herein by reference in its entirety.

### TECHNICAL FIELD

The present disclosure relates generally to systems and methods for assessing the status of a subject undergoing respiratory therapy using a mechanical ventilator. In particular, systems and methods are introduced that provide assessment and predictive analysis for a future response of a patient after the extubation procedure.

### BACKGROUND

Thousands of children and adults are admitted to intensive care units each year and placed on mechanical ventilators. Although it has been over forty years since the first pediatric ventilator was designed, there has been no specific method for cardiopulmonary directed therapy that has proven superior to individual clinical decision related to extubation. While mechanical ventilation is generally lifesaving, prolonged use of a mechanical ventilator may lead to a number of adverse conditions. Accordingly, timely liberation from mechanical ventilation may be critical to avoiding or mitigating the adverse effects of prolonged use of a mechanical ventilator.

Thus far, clinical decisions regarding the administration of a mechanical ventilator have relied heavily on the clinical judgment of the health care provider overseeing the therapy, typically, without a standardized approach to measurements of a patient's pre-extubation cardiopulmonary status. Previously, past studies have concluded that attempts at standardized approaches such as extubation readiness tests, have not proven superior to human decision-making Thus, current protocols entail the provider making intermittent and subjective assessments of the patient's condition. For example, a health care provider may periodically assess the patient's physiological condition and make adjustments to the ventilation therapy as needed (e.g., by determining when to extubate the patient, when to adjust the ventilator's settings, etc.). While overall rates of reintubation or noninvasive ventilation following extubation are relatively low, an increase in noninvasive ventilation (NIV) is seen particularly in more complex patients who have pre-existing respiratory or neurologic conditions.

Some attempts have been made to standardize treatment protocols and guidelines concerning extubation of a patient from a mechanical ventilator, but treatment approaches across the industry still remain widely inconsistent. For example, an “extubation readiness test” based on what was previously proposed by Randolph et al may be implemented. Typically, there are minimal settings on which patients must maintain their SpO2 and VT/kg over a period of time. However, this approach and others have largely failed to predict extubation success and this extubation assessment is not validated for some of the most complex patients, such as those with neuromuscular disease. Further, it is not designed to predict need for NIV. In particular, studies have shown that the extubation of a patient from mechanical ventilation therapy remains inconsistent and is often left to the best judgment of the health care provider. To date, clinical decisions regarding mechanical ventilation and extubation largely have relied on intermittent assessments of physiologic parameters, without the ability to effectively integrate complex data regarding pulmonary mechanics, gas exchange, and cardiopulmonary interactions over time.

### SUMMARY

Advantageously, the exemplary embodiments of the present invention allow for the assessment and predictive analysis of a patient undergoing respiratory therapy using a mechanical ventilator regarding a future response after the extubation procedure. In one aspect an exemplary embodiment of the present invention, a method for determining patient response to an extubation procedure may include one or more physiologic, pulmonary mechanism or oxygenation-related parameters measurements for a predetermined period of time that may be received and a known data set may be retrieved. A first extubation index with the physiologic or pulmonary mechanism measurements may be calculated and the calculation may be compared with the known data set. Accordingly, an assessment that concerns the utilization of a device after extubation may be produced.

In some exemplary embodiments the physiologic measurements may include at least one of the following an oxygenation parameter, a ventilation parameter, or a pressure parameter. The pulmonary mechanism measurements may include at least one of the following a measured heart rate, a measured blood pressure of the patient, a rate pressure product value, or a pulse pressure value.

In other exemplary embodiments, the predetermined period of time may be greater than two hours. Additionally, in some exemplary embodiments, the predetermined period of time may be less than three hours. In some exemplary embodiments, the method may include at least one of a plurality of the physiologic, pulmonary mechanism measurements or oxygenation-related parameters may be simultaneously received and analyzed. In another exemplary embodiment, at least one of a plurality of the physiologic, pulmonary mechanism measurements or oxygenation-related parameters may be continuously updated and the calculation may be continuously updated. Further, the one or more physiologic or pulmonary mechanism measurements may be continuously updated.

The known data set may further include evaluating a history of measurements and outcomes to detect a potential post extubation condition that may be evaluated and the detected post extubation condition to a user interface may be reported. For example, a patient may be compared to a cohort of patients by a variety of parameters (e.g., disease, age, event and procedure). Furthermore, a second extubation index with the physiologic or pulmonary mechanism measurements may be calculated. The first extubation index calculation may be compared to the second extubation index to predict the utilization of a device after extubation.

In another aspect of an exemplary embodiment, a system for predicting a state of a subject after an extubation procedure from a mechanical ventilator, may include one or more network interfaces that may communicate with a network, a processor may be coupled to the one or more network interfaces and may be configured to execute one or more processes; and a memory may be coupled to store a process executable by the processor. The process when executed may be operable to receive at least one physiologic, pulmonary mechanism or oxygenation-related parameters measurements regarding the subject and may compare a known data set to the at least one physiologic, pulmonary mechanism or oxygenation-related parameters measurements. A predictive device utilization assessment for a subject after the extubation procedure may be predicted.

In some exemplary embodiments, the system may include at least one physiologic, pulmonary mechanism or oxygenation-related parameters measurements regarding the subject that may be continuously updated from the network. In another exemplary embodiment, the predictive device utilization assessment for a subject after extubation may be continuously updated. The system may include the predictive device utilization assessment for a subject after extubation that may be continuously updated after at least one physiologic, pulmonary mechanism or oxygenation-related parameter measurement may be updated.

The system may include that the process when executed may be further operable to evaluation a history of subject measurement conditions and operating conditions of the ventilator to detect a potential subject condition and report the detected subject condition to the user interface. In some exemplary embodiments, the process when executed may be further operable to receive at least one of a static or dynamic compliance per KG, an oxygen saturation (e.g., arterial, venous or from pulse oximetry), NIRS, a fraction of inspired oxygen, PaO2, a PEEP level, PIP level, mean airway pressure, respiratory rate, CO2 level (e.g., from either end tidal, transcutaneous, or pCO2 from a venous, arterial or capillary blood gas) and a tidal volume. Furthermore, the system may include that when the process may be fully executed at least one of a static or dynamic compliance per KG, an oxygen saturation, a fraction of inspired oxygen, a PEEP level, PIP level, mean airway pressure, respiratory rate CO2 level (e.g., from either end tidal, transcutaneous, or pCO2 from a venous, arterial or capillary blood gas) and a tidal volume or a mean airway pressure may be received.

In another exemplary embodiment, a non-transitory computer readable medium containing program instructions executed by a processor, the programming instructions may include program instructions that may receive one or more physiologic, pulmonary mechanism or oxygenation-related parameters measurements for a predetermined period of time. The program instructions may retrieve a known data set and may calculate a first extubation index with the physiologic or pulmonary mechanism measurements. Furthermore, the program instructions may compare the calculation with the known data set; and may produce an assessment of a device after extubation. In some embodiments, the non-transitory computer readable medium may further include program instructions that may calculate a second extubation index with the physiologic or pulmonary mechanism measurements.

The additional features of the present disclosure will be described infra.

### BRIEF DESCRIPTION OF THE DRAWINGS

The embodiments herein may be better understood by referring to the following description in conjunction with the accompanying drawings in which like reference numerals indicate identically or functionally similar elements, of which:

FIG. 1 illustrates an example computer system;

FIG. 2 illustrates an example network device for categorizing a state of a subject undergoing therapy from a mechanical ventilator;

FIG. 3A illustrates an exemplary graphical representation exemplary ROC curves for predictive analysis regarding a future response of a patient categorized in the NIV group after the extubation procedure;

FIG. 3B illustrates an exemplary graphical representation exemplary ROC curves for predictive analysis regarding a future response of a patient categorized in the Reintubation group after the extubation procedure;

FIG. 3C illustrates an exemplary graphical representation exemplary ROC curves for predictive analysis regarding a future response of a patient categorized in the Combined Device group after the extubation procedure; and

FIG. 4 illustrates an exemplary graphical representation of dynamic compliance from 2.5 hours prior to extubation for the response groups including the no device group, non-invasive group, reintubation group and the combined group;

### DESCRIPTION OF EXEMPLARY EMBODIMENTS

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

As used herein, the term “subject” is meant to refer to an animal, preferably a mammal including a non-primate (e.g., a cow, pig, horse, cat, dog, rat, mouse, etc.) and a primate (e.g., a monkey, such as a cynomolgus monkey, and a human), and more preferably a human. For example, in a hospital or other clinical setting, a subject may otherwise be referred to as a patient.

Furthermore, the control logic of the present invention may be embodied as non-transitory computer readable media on a computer readable medium containing executable program instructions executed by a processor, controller or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

Illustratively, the techniques described herein are performed by hardware, software, and/or firmware, which may contain computer executable instructions executed by the processor 220 (or independent processor of interfaces 210) to perform functions relating to the techniques described herein, e.g., in conjunction with communication process 244. For example, the techniques herein may executed on an aggregate of servers over wireless communication protocols, and as such, may be processed by similar components understood in the art that execute those protocols, accordingly.

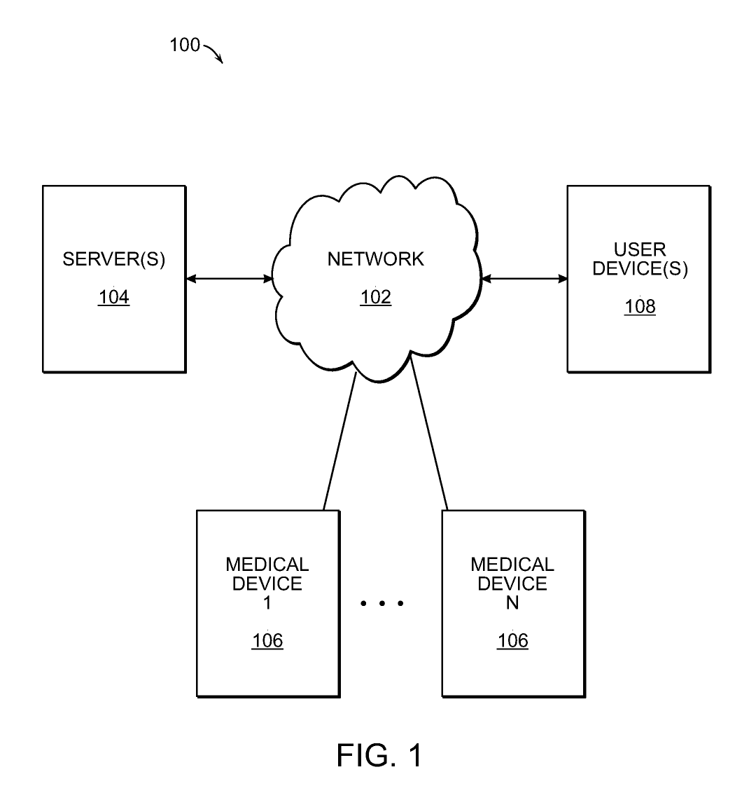

FIG. 1 is a schematic block diagram of an example computer system 100 comprising any number of user devices 108, servers 104, and/or medical devices 106 interconnected by various methods of communication which are illustratively represented as network 102. For instance, communication between the devices via network 102 may be over wired links or via a wireless communication medium (e.g., WiFi, cellular, etc.), where certain devices may be in communication with other devices based on distance, signal strength, current operational status, location, etc. Those skilled in the art will understand that any number of devices, links, etc. may be used in system 100, and that the view shown herein is for simplicity. Also, while a single network 102 is shown, network 102 may comprise any number of public or private network and/or direct connections between the devices.

According to various embodiments, medical devices 106 (e.g., a first through nth medical device) may include a ventilator (e.g., a mechanical ventilator), any sensors associated therewith (e.g., an airway pressure sensor, etc.), and any number of devices that monitor or otherwise collect/process data regarding the physiological condition of a subject undergoing therapy using the ventilator. For example, medical devices 106 may also include, but are not limited to, cardiovascular monitors (e.g., a heart rate monitor, a blood pressure monitor, etc.), any number of carbon dioxide (CO₂) sensors (e.g., an end tidal CO₂ monitor, a transcutaneous CO₂ monitor, a blood gas analyzer, etc.), any number of oxygen (O₂) sensors (e.g., a pulse oximeter (SPO2), a near infrared spectroscopy (NIRS) analyzer, a venous oximetry (SVO2) detector, a blood gas analyzer), etc.

Servers 104 may collect data from the one or more medical devices 106. The collection may be made either on a push basis (e.g., a particular medical device 106 sends data to a particular server 104 without first receiving a request to do so) or on a pull basis (e.g., the device 106 provides the data only after receiving a request for the data from server 104). The data received by servers 104 may include any data from medical devices 106 regarding the status of a subject undergoing respiratory therapy using a ventilator and/or operating parameters of the ventilator itself. Servers 104 may store the received data and may make any number of computations using the received data. For example, servers 104 may calculate any number of statistics (e.g., an average measurement, a maximal or minimal measured value, etc.) using the received data. In one embodiment, servers 104 may compute any number of values based on the received data. For example, servers 104 may compute a fraction of inspired oxygen (FIO2) value, an O₂ saturation to FIO2 ratio, etc., if not already calculated by medical devices 106 and included in the data received from medical devices 106. In another embodiment, servers 104 may compute trends using the data received from medical devices 106. For example, servers 104 may compute a moving average, estimated/predicted value, or the like based on a history of the data received from medical devices 106.

User device(s) 108 may include any device configured to convey or receive sensory input to and/or from a user. For example, user device(s) 108 may include, but are not limited to, personal computers, tablet devices, smart phones, smart watches, other wearable electronic devices, personal digital assistants (PDAs), or the like. In some case, user device(s) 108 may receive data from servers 104 and/or medical device 106. For example, servers 104 may provide a webpage interface to a particular user device 108 that displays data regarding the status of a patient to the user (e.g., current measurements or calculations, trends, alerts, etc.). In some embodiments, user device(s) 108 may be operable to provide data to servers 104 and/or to medical devices 106. For example, a web-based interface served by servers 104 may be configured to receive annotations or other manually entered data regarding the patient (e.g., lab results, demographics information, medical history information, etc.).

As would be appreciated, any of the functions described herein with respect to servers 104, medical devices 106, and user devices 108 may be performed in a distributed manner across the various devices or integrated into a singular device, in various embodiments. For example, while certain functions are described herein with respect to servers 104, these functions may alternatively be performed by any of medical devices 104 or user device(s) 108.

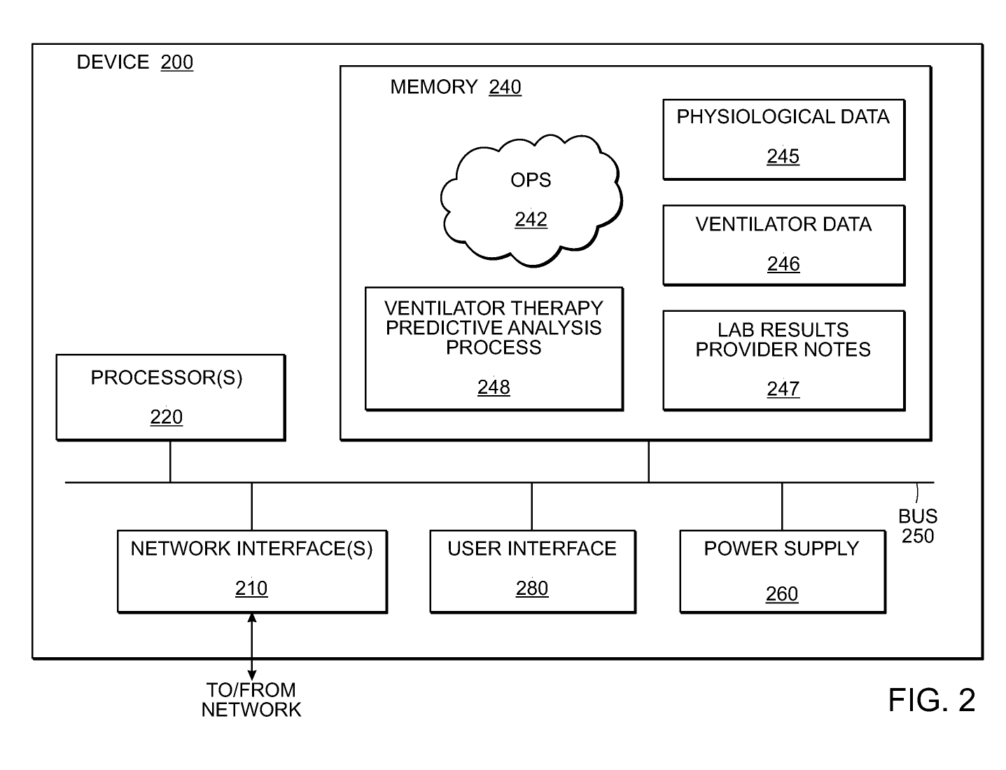

FIG. 2 is a schematic block diagram of an example device 200 that may be used with one or more embodiments described herein, e.g., as any of devices 104-108 shown in FIG. 1. The device may include one or more network interfaces 210, one or more user interfaces 280 (e.g., an electronic display, a speaker, a microphone, a keypad, etc.), at least one processor 220, and a memory 240 interconnected by a system bus 250, as well as a power supply 260 (e.g., battery, plug-in, etc.).

The network interface(s) 210 contain(s) the mechanical, electrical, and signaling circuitry for communicating data over physical and/or wireless links coupled to the network 102. The network interfaces may be configured to transmit and/or receive data using a variety of different communication protocols, including, inter alia, TCP/IP, UDP, wireless protocols (e.g., IEEE Std. 802.15.4, WiFi, Bluetooth®), Ethernet, etc. Namely, one or more interfaces may be used to communicate with the user on multiple devices and these interfaces may be synchronized using known synchronization techniques.

The memory 240 may include a plurality of storage locations that are addressable by the processor 220 and the network interfaces 210 for storing software programs and data structures associated with the exemplary embodiments described herein. As noted above, certain devices may have limited memory or no memory (e.g., no memory for storage other than for programs/processes operating on the device). The processor 220 may comprise necessary elements or logic configured to execute the software programs and manipulate the data structures, such as physiological data 245, ventilator data 246, and/or lab results provider notes and targets or goals of the therapy 247. An operating system (OPS) 242, portions of which are typically resident in memory 240 and executed by the processor, functionally organizes the device by, inter alia, invoking operations in support of software processes and/or services executing on the device. The processes and/or services may include a ventilator therapy predictive analysis process 248, as described herein.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules configured to operate in accordance with the techniques herein (e.g., according to the functionality of a similar process). Further, while the processes have been shown separately, those skilled in the art will appreciate that processes may be routines or modules within other processes.

Ventilator therapy predictive analysis process 248 may contain computer executable instructions executed by the processor 220 to perform the various functions described herein regarding predictive analysis of the future condition of a subject undergoing respiratory therapy using a mechanical ventilator after an extubation procedure from the respiratory therapy. In particular, ventilator therapy predictive analysis process 248 may analyze physiological data 245 (e.g., received data regarding the physiological condition of the subject), ventilator data 246 (e.g., received data regarding the settings or operation of the ventilator itself), and/or data 247 that may include lab results or provider notes (e.g., digitized notes from a healthcare provider, laboratory results regarding the subject, etc.), to analyze or categorize the expected future status of the subject.

In some embodiments, ventilator therapy predictive analysis process 248 may also contain instructions that generate future treatment protocols for physician review based on the predicted future state of the subject and provide such treatment protocols to user interface 280 or to a user interface of another device (e.g., via network 102). For example, ventilator therapy predictive analysis process 248 may receive data from a bedside monitor, mechanical ventilator, digitized laboratory reports, radiology reports, an intravenous (IV) pump, an intracranial pressure (ICP) monitor, etc., and aggregate the data to analyze the condition of the subject. Based on the aggregated data, ventilator therapy predictive analysis process 248 may determine that the condition of the subject falls within a pattern and the patient may likely exhibit a particular response to an extubation procedure and, in response, provide a corresponding treatment protocol to a user interface device for physician review.

### Data Collection and Assessment

Ideally, predictive assessments may include both a robust data collection system together with an approach to analysis that may provide insight for specific therapeutic interventions in a data rich environment. In some embodiments, physiologic monitoring and ventilator software may provide the capability to stream or export information in real time in a digital data format, which may enabled the coupling of high frequency data sampling with near real-time analysis.

In one exemplary embodiment, data may be collected on patients undergoing therapy with a mechanical ventilation system. In particular, the ventilator therapy predictive analysis process may obtain data on the pulmonary response and/or any parameters available from the ventilator. For example, the ventilation category may be based in part on a lung compliance measurement, a lung resistance measurement, a lung elastance measurement, a minute ventilation value, a flow value, a volumetric CO₂ (VCO2) measurement, a mean airway pressure, a tidal volume (Vt) value from the ventilator, an end tidal CO₂ (ETCO₂) measurement, a respiratory rate (RR), a flow rate, a circuit pressure, a calculated measurement (e.g., ventilation index (Vd/Vt) elimination over one minute), combinations thereof, or any other calculation or value available regarding the status of the subject and/or ventilator. Furthermore, cardiovascular measurements may be obtained from a patient undergoing mechanical ventilation therapy and may include, but are not limited to, a cardiac output (e.g., either a Fick calculation or a direct measurement), a measured heart rate (HR), a measured blood pressure (BP), a calculated rate pressure product (RPP) where RPP=HR*BP, or a calculated pulse pressure (e.g., the difference between systolic and diastolic BP measurements). Additionally, in various exemplary embodiments, oxygenation-related parameters may include data produced from a calculated ratio of the oxygen saturation (SpO2) of the subject to the fraction of inspired oxygen (FIO2), also known as an S/F ratio (i.e., S/F=SpO₂-/FIO2). The oxygenation-parameters may also include an oxygen saturation index (OSI) calculated as the mean airway pressure (MAP)*FIO2/SpO₂.

In addition to the ventilation and oxygenation categories described above, data collection and assessment may also assess the physiological and ventilator data, to identify a number of specific conditions of a subject undergoing ventilator therapy. In various embodiments, these conditions may include, but are not limited to, acute respiratory distress syndrome (ARDS), ventilator associated lung injury (VALI), the subject requiring extubation readiness testing (ERT), a ventilator associated event (e.g., adults) and condition (e.g., pediatrics) (VAE/VAC) as defined by the U.S. Center for Disease Control (CDC), or the subject being ready for extubation. In an exemplary embodiment, the patient's physiological and ventilator data, may be applied to a prospective and/or random data collection assignment with retrospective analysis. In particular, patients anticipated to require invasive mechanical ventilation for longer than 3 hours may be selected to have their ventilator and physiologic monitors connected a data collection technology system (e.g., tracking, trajectory, and triggering decisions platform). The data collection technology system may aggregate, store, and display comprehensive real-time patient data for clinicians. The data collection technology system technology may provide a platform to track vital patient data on a manipulatable monitoring system viewable on standard web-browsers commonly found within a hospital environment, and may host a plurality of research evaluation criteria.

The data collection technology system may include a user interface that may enable clinicians to explore and analyze a patient's physiologic data sets including both instantaneous values and extrapolated as a time series. For example, data streams may be displayed across the central region of the user interface, with all available data streams represented in an integrated panel. Further, multiple data streams may be visualized simultaneously and may be arranged to be configurable to be displayed data in different orders or overlaid upon each other. The time window may be expanded or contracted for rapid visualization of a patient's current status or historical course of treatment. In particular, data target ranges may be specified to provide prompt visualization of anomalous or undesirable physiologic values. Annotations, events, and decision data may be inserted at specified time points. Still further, the data collection technology system may capture and display data collected by bedside monitors, mechanical ventilators, Admission/Discharge/Transfer (ADT) systems, a local clinical annotation system or other data collection devices.

Further, to evaluate the usefulness of the mathematical model with the aforementioned characteristics, two clinically derived models: The extubation readiness test (ERT Score—which is the standard of care in a PICU and its application remains largely based on clinical judgment), and a model based on the modified integrative weaning index developed by clinical experts (EX Score) may be compared. The ERT Score may be evaluated during the preceding two hours of each point in time during which the patient was within the desired test-thresholds (ready for extubation). Then, the percentage of time during which the test's criteria met as a score of 0-1 (0%-100% extubation readiness) may be calculated. The EX Score may be calculated at each time point and the mean of these results are used for the final prediction at the point of extubation.

### Categorization of Predicted Future Response Post-Extubation

According to an exemplary embodiment, an assessment of a real time (e.g. continuously updating) dataset obtained from a patient may produce a real time (e.g., continuously updating) assessment of a patient's predicted future response post extubation. As shown in Table 1 below, the extubation status of patients may be categorized into a number of different categories based on the maximum support revived within twenty four hours of extubation. These categories and their corresponding criteria are shown below, according to various embodiments:

TABLE 1

| Extubation Status | Device Determination |
|---|---|
| No Device | Not requiring any support other than simple |
| | oxygen therapy |
| NIV | Required CPAP or BiPAP |
| Reintubation | Required the endotracheal tube to be replaced |

In some exemplary embodiments, in order to produce an assessment, a patient's data may be collected 3 hours prior to extubation and may be analyzed and compared to the device utilization extubation indices as discussed below. Following initial analysis, data 30 minutes prior to extubation may be excluded due to artifacts surrounding extubation preparation, such as suctioning, deflation of the cuff or removing of the tape. The remaining data may include approximately 2.5-hour duration of data for inclusion within the analysis period.

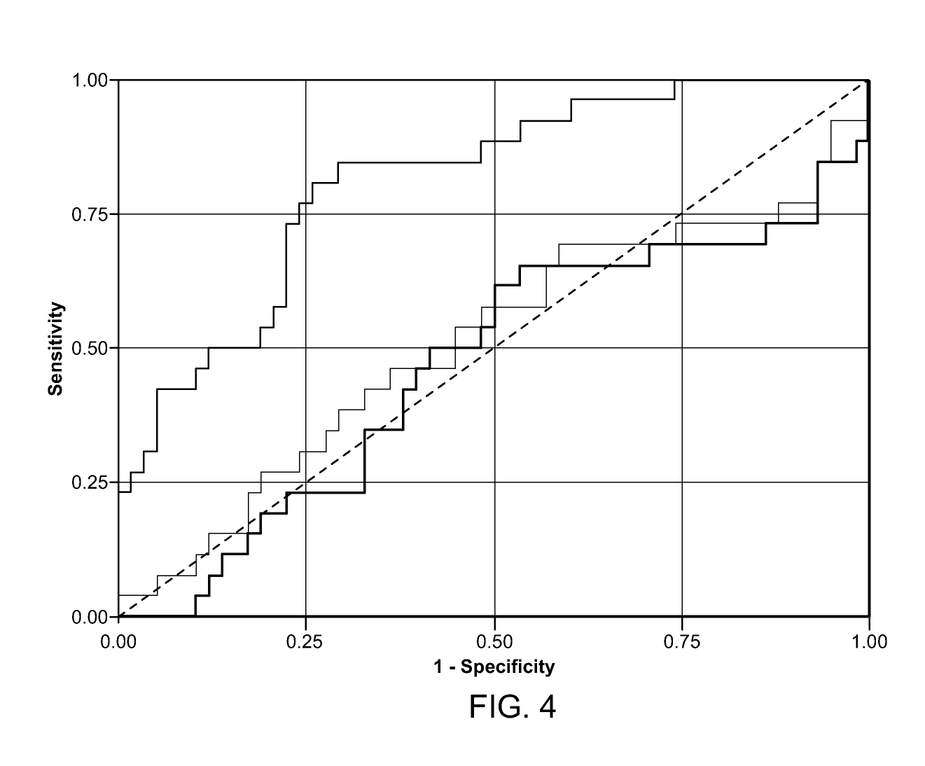

In some exemplary embodiments, data may be collected from the mechanical ventilator and physiologic monitors at about 5 seconds increments. In particular, means and standard deviations may be calculated based on parameters according to absolute number (e.g, ex. FIO₂) or specific information (e.g., age or weight) such as tidal volume or respiratory rate (e.g., RR) and identified by grouping. The “no device” status as the best outcome may be used as the baseline comparison. Further, Kruskal-Wallis one-way analysis of variance may be the non-parametric method used for small and differing sample sizes. Additionally, Receiver Operating Characteristics (e.g., ROC) curve may be performed to illustrate the performance of the selected parameters or newly developed indices. For example, as shown in FIGS. 4A-4C, one curve may be provided for each outcome. The area disposed under the curve may be provided for each curve within the selected graphs. In some embodiment's, Receiver Operating Characteristics (ROC) curve may be performed to illustrate the performance of the selected parameters or newly developed indices.

In some exemplary embodiments, three models may be derived from a similar set of variables. However, a different number of variables and different calculations may be applied for each model. The ERT score may include Vte, PEEP, FiO₂, PₑₜCO₂, SpO₂, and spontaneous RR (FIG. 1). The EX score may include a subset of these variables, using: Vt, oxygen saturation index (SI=(FiO₂×MAP)/SpO₂), as well as dynamic compliance (Cdyn), which is not part of the ERT score. The computer derived CDE model may use variables such as a maximum PEEP, maximum Cdyn, mean ventilation index (VI=(RR×(PIP-PEEP)×PaCO₂/1000), mean SI, and VCO₂. Further, “Tachypnea,” which is the percentage of time that the patient had a fast or slow RR based on their age. Higher “Tachypnea” values mean that the patient was tachypneic during a greater percentage of the time in the 3 hours preceding extubation), may be considered.

### Extubation Index

After generating the patient data points over the predetermined duration, and establishing the known categorization criteria for patients post extubation, the extubation indices may be applied to the patient data set. In, particular, two extubation indices developed were based on the modified integrative weaning index, parameters trending towards significance, and expert opinion of clinically relevant parameters such as FIO₂ and MAP and Cdyn. The assessment data sets may be extrapolated and applied to the first or the second extubation index. In some exemplary embodiments, extubation indices may be compared to each other and Cdyn alone may predictor the need for NIV and/or reintubation.

### First Extubation Test Index (ERT)=

### TVexp>=5;

### PEEP<8;

### FiO2<50;

### SpO2>94;

### Spontaneous Respiratory Rate

If (age>5) return (RR>12 & RR<30);
If (age>2 & age<=5) return (RR>15 & RR<45);
If (age>0.5 & age<=2) return (RR>25 & RR<45);
If (age<=0.5) return (RR>30 & RR<55);
Tachycardia/Bradycardia & Tachypnea/Bradypnea are the precentage of time that the patient was with fast or slow HR or RR based on their age. Higher Tachpnea values indicate that the patient was tachypneic during a greater percentage of the time in the 3 hours preceding extubation.

### Second Extubation Index (Expert Derived Model)

=[Cdyn÷(FiO₂×MAP÷SpO₂)]×(f÷VT)×100

### Computer Derived Equation (CDE—Mathematical Model)

4.846*(Intercept)+−174.628*I(1/I(PEEP_MAX̂2))+9.695*I(Tachypnea)+−0.098*I(Cdyn_MAX*VI_MEAN)+1.479*I(SI_MEAN*VCO2_f_MEAN)

Additionally, in an alternate embodiment, calculations may include and be compared to the ventilation index as a measurement of extubation readiness.

$$VI = \frac{RR \times ( PIP - PEEP ) \times Pa {CO}_{2}}{1000}$$

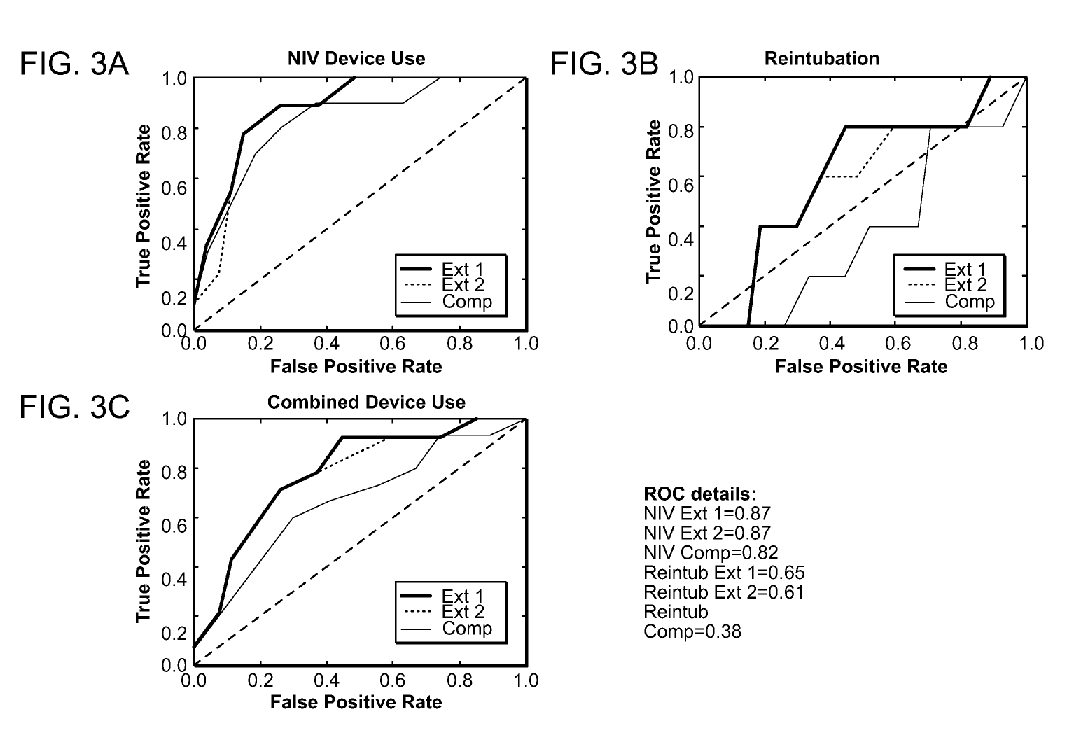

As shown in exemplary graphical representations FIGS. 3A-3C the ROC curves may be generated for the first extubation index 1=(Cdyn× S/F)÷f/VT and the second extubation index=[(Cdyn÷SOI)×f/VT]×100, where SOI=[FIO₂×MAP÷SPO₂]×100; Cdyn for each extubation status characterization group.

For example FIG. 3A is an exemplary graphical representation of the curve generated from the data for the NIV device group. FIG. 3B illustrates an exemplary graphical representation for the curves for the data set of the group requiring reintubation. Further, FIG. 3C illustrates a graphical representation for the combined treatment group.

According to an exemplary embodiment, extubation indices may predict the need for NIV support after extubation better than Dynamic compliance per Kg (Cdyn) alone. Furthermore, the data generated from historical assessments of patients who did not require device support post extubation comprise the no device group reference or baseline dataset for all comparisons. In an exemplary embodiment, the ROC curves for Cdyn as well as both indices in the NIV group, the reintubation group and the combined group may be generated. Based on the ROC curves, both indices as well as dynamic compliance, the need for non-invasive use may be predicted. In some exemplary embodiments, the first and second extubation indices may produce a ROC for the NIV group of 0.87, compared to 0.82 produced by dynamic compliance alone. As shown in FIG. 4 the Cdyn values may be produced over the 2.5 hour duration prior to extubation. For, example patients that may require NIV support may have a consistently lower Cdyn than patients classified within the alternate categories.

Based on the ability of Cdyn to predict need for NIV ventilation and other commonly associated factors such as indices of oxygenation such as FIO2, MAP and SpO2, two extubation indices may be produced, which incorporate Cdyn with other proposed markers of extubation readiness.

### CONCLUSION

The use of continuous physiologic and pulmonary mechanics data prior to liberation from mechanical ventilation may delineate subtle differences not often realized or documented in those who will require additional support following extubation. For example, when considering the NIV group, the data assessment may generally present a data characterization that indicates that the rapid shallow breathing index per kilogram (f/VT/Kg) and Spontaneous respiratory rate (SpRR) was higher and the SpO2 was lower in the noninvasive ventilation (NIV) group (p<0.05). However, 3 parameters out of 14 parameters reach statistically significant differences.

### Experimental Results

An assessment utilizing the extubation prediction system was conducted. In particular, near continuous data (e.g., five second sampling) in which the one minute median result were applied to the two rules based algorithms to detect clinically relevant trends. The created first and second extubation success indices were able to predict the need for NIV support after extubation consistent with the standard of practice. The third model was developed to improve upon the first two models by using a computer derived extubation prediction.

The three models were derived from a similar set of variables, but a different number of variables and different calculations were applied for each model. The ERT score used variables that include Vte, PEEP, FiO2, PetCO2, SpO2, and spontaneous RR (FIG. 1). The EX score included a subset of these variables, using: Vt, oxygen saturation index (SI=(FiO2× MAP)/SpO2), as well as dynamic compliance (Cdyn), which is not part of the ERT score (FIG. 2). The computer derived CDE model used variables including maximum PEEP, maximum Cdyn, mean ventilation index (VI=(RR×(PIP-PEEP)×PaCO2)/1000), mean SI, and VCO2. We also included “Tachypnea,” which is the percentage of time that the patient had a fast or slow RR based on their age. Higher “Tachypnea” values mean that the patient was tachypneic during a greater percentage of the time in the 3 hours preceding extubation, please see FIG. 3.

Mathematical Model (CDE Score)

To prepare the data for the mathematical multivariate model, the 180 observations associated to every variable (e.g., one-minute sampling rate for three hours, 180 minutes) were transformed into a list of five summary statistics per variable that included the minimum value of such variable within the observed time period, the maximum value, the mean, median, and standard deviation. The 65 summary statistics (e.g., 5 summary statistics×13 variables) per patient were combined with variables obtained from the electronic medical records (EMR) and clinical notes to provide a single collection of data points for each of the 89 extubation cases. The variables obtained from the EMR and clinical notes included age, total hours on ventilator, and the extubation outcome.

A collection of multivariate models were built using a logistic regression approach with step-wise forward selection of variables, starting with a single variable and adding each time one additional variable that will bring the most significant improvement for the model, and mapping the 65 summary statistics to extubation outcome. For a fair comparison with the other extubation indices, and in order to minimize over-fitting, model exploration was limited to models that use at most three input variables. The models were allowed to select non-linear and interaction terms as input variables.

To evaluate the usefulness of the best performing mathematical model with the aforementioned characteristics, the model was compared to two clinically derived models. First the extubation readiness test (e.g., ERT Score—which is the standard of care in our PICU and its application remains largely based on clinical judgment), and (2) a model based on the modified integrative weaning index that was developed by clinical experts at the study's site (EX Score). The ERT Score evaluated the amount of time during the preceding two hours of each point in time during which the patient was within the desired test-thresholds (e.g., ready for extubation). Then the percentage of time during which the test's criteria were met as a score of 0-1 (0%-100% extubation readiness) was calculated. The EX Score was calculated at each time point and the mean of these results was used for the final prediction at the point of extubation

Furthermore the classifications were probabilistically summarized by calculating the percent of time a subject belongs to a category. A total of 104 patients were screened for this analysis. Fifteen (14.5%) subjects were excluded; these patients were proportionally distributed among the study groups. Exclusion reasons included insufficient data, characterized by <50% of required data (5 patients), death (5 patients), discharged on support (4 patients), inability to collect data due to maintenance of patient monitoring infrastructure and an extremely abnormal physiologic variable likely due to monitor artifact (1 patient). Ultimately, 80 mechanically ventilated patients aged 1 day to 32 years (mean 6.6) were enrolled with 89 associated extubation attempts. Among the 89 total extubation attempts, in 57 (64%) cases the patient was extubated without requiring any supportive device other than simple oxygen therapy, in 23 (25.8%) cases the patient required NIV, and in 9 (10.1%) cases the patient required reintubation.

Selected ventilator and physiologic parameters during the three hour sampling period are summarized in Table 2 based on post-extubation outcome. Using the no support group as the referent for all comparisons, the rapid shallow breathing index per kilogram f/Vt*kg⁻¹ was higher in the NIV group (5.53 vs. 4.02, p=0.001). Patients who required NIV also had statistically higher respiratory rate (24.0 vs. 18.7, p=0.03) and lower SpO₂ (97.9 vs. 98.8, p=0.04) than the no support group.

TABLE 2

| Variable | No Support | NIV | Reintubation |
|---|---|---|---|
| Cdyn | 0.82 [0.75-0.89] | 0.74 [0.64-0.84] p = | 0.97 [0.54-1.4] p = 0.401 |
| | | 0.195 | |
| SpO2 | 98.76 [98.42-99.1] | 97.91 [97.13-98.69] p = | 99.19 [97.75-100.64] p = |
| | | 0.043 * | 0.373 |
| FiO2 | 33.28 [31.63-34.93] | 36.75 [33.31-40.19] p = | 35.6 [27.56-43.64] p = |
| | | 0.106 | 0.492 |
| RRaw | 21.05 [18.85-23.26] | 25.02 [20.21-29.83] p = | 22.85 [12.59-33.1] p = |
| | | 0.117 | 0.559 |
| SpRR | 18.65 [16.07-21.23] | 24.04 [19.03-29.06] p = | 22.75 [12.58-32.91] p = |
| | | 0.03 * | 0.286 |
| TVexp | 7.3 [6.76-7.84] | 6.82 [5.94-7.7] p = | 9.46 [6.91-12.01] p = |
| | | 0.343 | 0.027 * |
| MAP | 7.65 [7.38-7.93] | 7.96 [7.44-8.47] p = | 8.32 [6.75-9.9] p = 0.162 |
| | | 0.433 | |
| HR | 101.13 [95.09-107.16] | 109.7 [99.76-119.64] | 129.43 [88.75-170.12] p = |
| | | p = 0.23 | 0.05 |
| PIP | 14.28 [13.62-14.95] | 14.85 [13.74-15.97] p = | 15.12 [12.58-17.66] p = |
| | | 0.353 | 0.309 |
| PEEP | 4.81 [4.67-4.94] | 5.07 [4.82-5.32] p = | 5.01 [4.26-5.76] p = 0.576 |
| | | 0.069 | |
| TVin | 183.66 [146.24-221.09] | 190.15 [124.63-255.67] | 117.66 [60.05-175.26] p = |
| | | p = 0.977 | 0.525 |
| etCO2 | 43.08 [41.13-45.02] | 44.14 [42.08-46.21] p = | 42.85 [36.48-49.22] p = |
| | | 0.479 | 0.758 |
| VCO2 | 3.72 [3.01-4.42] | 3.95 [2.93-4.98] p = | 1.45 [4.52-4.42] p = |
| | | 0.829 | 0.221 |
| SpO2/FiO2 | 322.39 [304.54-340.24] | 294.01 [266.75-321.28] | 292.02 [228.28-355.76] |
| | | p = 0.131 | p = 0.666 |
| f/Vt * kg−1 | 4.02 [3.01-5.02] | 5.53 [3.82-7.23] p = | 2.73 [1.19-4.26] p = 0.492 |
| | | 0.01 * | |
| SI | 2.6 [2.42-2.77] | 3.01 [2.59-3.43] p = 0.1 | 3.03 [1.82-4.24] p = 0.321 |
| VCO2/f | 0.19 [0.16-0.23] | 0.19 [0.14-0.25] p = | 0.07 [−0.06-0.2] p = 0.072 |
| | | 0.712 | |
| VI | 13.13 [11.45-14.81] | 16.35 [12.55-20.14] p = | 15.16 [6.19-24.12] p = |
| | | 0.093 | 0.462 |

Table 2: Data prior to extubation. The 30 minutes prior to extubation were eliminated due to artifact related to the extubation procedure such as suctioning, leak test, tape removal, and manual ventilation. All data are the mean value during this time period. P values are calculated based on the no support (referent) group. ‘Cdyn’, ‘TVexp’, ‘VCO2’ are scaled by weight.

The no support group versus those patients who received unplanned support after extubation (either unplanned NIV or unplanned re-intubation) were compared. The AUC for the clinically based ERT Score=0.54 [0.37-0.72], EX Score=0.62 [0.45-0.78], and the CDE Score AUC=0.65 [0.47-0.83]. With 10% false-positive rate, the CDE identified 36% of these unplanned-support cases compared to only 8% and 7% for the ERT and EX models (Table 2). We likewise tested the no support group versus those who had planned support. The AUC for the clinically based ERT Score=0.54 [0.37-0.71], EX Score AUC=0.53 [0.35-0.71], and the multivariate model CDE Score AUC=0.79 [0.64-0.93].

For example, as shown below in Table 3 the three studied models in predicting the represent the need for non-invasive support. Two indices were developed based on current practice; extubation readiness testing (ERT Score), and expert opinion that incorporated oxygenation and compliance indices (EX Score). A third computer derived extubation (CDE Score) was derived from a mathematical model. The accuracy is defined as the overall percentage of correct predictions. The CDE score provided the most improved performance of all three.

TABLE 3

| | | | | | |
|---|---|---|---|---|---|
| Accuracy | Sensitivity | Specificity | PPV | NPV | AUC |

No Support vs. Non-Invasive Support

| | | | | | | |
|---|---|---|---|---|---|---|
| ERT | 0.63 | 0.08 | 0.90 | 0.28 | 0.67 | 0.54 |
| EX | 0.66 | 0.12 | 0.90 | 0.34 | 0.69 | 0.50 |
| CDE | 0.75 | 0.42 | 0.90 | 0.65 | 0.78 | 0.72 |

No Support vs. Any Unplanned Support

| | | | | | | |
|---|---|---|---|---|---|---|
| ERT | 0.73 | 0.08 | 0.9 | 0.16 | 0.79 | 0.54 |
| EX | 0.74 | 0.07 | 0.9 | 0.15 | 0.80 | 0.62 |
| CDE | 0.79 | 0.36 | 0.9 | 0.46 | 0.85 | 0.65 |

As shown in table four below, the 2.5 hours prior to extubation are presented, in conjunction with the time based on age related parameters. The statistically significant finding included a higher respiratory rate in a single age group of reintubation category (p<0.05).

TABLE 4
Measurements 2.5 Hours Prior To Extubation, by Age Category

| | | | | | Combined |
|---|---|---|---|---|---|
| | | No Device | Non-invasive | Reintubation | (NIV + Reintubation) |
| | | total n = 27 | total n = 14 | total n = 5 (n, | total n = 19 |
| | Parameter | (n, sd) | (n, sd) | sd) | (n, sd) |
| Heart Rate | <6 months | 135 (3, 13.5) | 125 (4, 21.1) | 139 (1, 0) | 128 (5, 19.6) |
| | 7 mo-2 yrs | 103 (9, 15.6) | 130 (2, 31.8) | 104 (1, 0) | 122 (3, 28.8) |
| | 3-5 yrs | 102 (6, 22.3) | — | 70 (1, 0) | — |
| | ≥6 yrs | 96 (9, 19.1) | 106 (4, 19) | 93 (2, 4.1) | 102 (6, 16.9) |
| Resp. Rate | <6 months | 31 (3, 7.2) | 41 (4, 16.3) | 43 (1, 0) | 41 (5, 14.6) |
| | 7 mo-2 yrs | 21 (9, 7.9) | 38 (2, 15.8) | 29 (1, 0) | 35 (3, 13.6) |
| | 3-5 yrs | 19 (6, 5.5) | — | 19 (1, 0) | — |
| | ≥6 yrs | 14 (9, 4.4) | 23 (4, 7.7) | 27 (2, 0.4)* | 24 (6, 6.5)* |
| Systolic | <6 months | 82 (3, 8.2) | 75 (2, 1.7) | — | — |
| Blood | 7 mo-2 yrs | 103 (9, 15.7) | 70 (1, 0) | 92 (1, 0) | 81 (2, 11.0) |
| Pressure | 3-5 yrs | 111 (6, 16.9) | — | 72 (1, 0) | — |
| | ≥6 yrs | 122 (9, 16.8) | 99 (2, 14.8) | 133 (1, 0) | 78.8 (3, 10.0) |
| Mean Blood | <6 months | 63 (3, 5.7) | 53 (2, 2.0) | — | — |
| Pressure | 7 mo-2 yrs | 77 (9, 11.6) | 55 (1, 0) | 61 (1, 0) | 58 (2, 2.9) |
| | 3-5 yrs | 80 (6, 20.4) | — | 61 (1, 0) | — |
| | ≥6 yrs | 82 (9, 9.9) | 73 (2, 5.3) | 92 (1, 0) | 79 (3, 10.0) |
| Diastolic | <6 months | 49 (3, 1.8) | 40 (2, 4.6) | — | — |
| Blood | 7 mo-2 yrs | 61 (9, 10.4) | 43 (1, 0) | 43 (1, 0) | 43 (2, 0.04) |
| Pressure | 3-5 yrs | 61 (6, 17.1) | — | 51 (1, 0) | — |
| | ≥6 yrs | 65 (9, 7.6) | 59 (2, 4.1) | 69 (1, 0) | 62 (3, 5.5) |

The analysis demonstrated that patients can be successfully categorized based on the goals of therapy. Moreover, coupling categorization with machine learning, resulting in real-time decision support could improve quality.

In further detail, the experimental approach consisted of first creating a clinical extubation score (ERT Score), to simulate clinical judgment (standard of care). This was based largely on previously published variables and commonly used physiologic metrics. Next, the EX Score was created, which incorporated calculations such as oxygen saturation index and Cdyn to better predict device utilization (expert opinion). This seemed logical as patients with poor pulmonary compliance would likely be at risk for needing support after extubation. The third phase was to create a mathematical model to predict device utilization in our cohort. Our CDE Score performed best with area under the curve (AUC)=0.8123. The model suggests that (a) the need for post-extubation device utilization is directly related to the level of PEEP at the time of extubation and (b) that large deviations in SpRR and high Cdyn is correlated with device-free extubation.

This analysis demonstrates the value of incorporating multiple physiologic measurements simultaneously as an adjunct to clinical judgment. It would not be plausible for a clinician to continually make these calculations in real time on multiple patients. Most often, the ability to predict outcome is reduced when calculations are simplified, as demonstrated with the clinical ERT score and EX Score. The fact that the CDE Score was able to greatly improve our ability to predict device utilization makes an argument in favor of the design of computer-based decision support systems capable of providing real-time calculations with a higher volume of more specific data.

Patients requiring NIV support following extubation had a significantly higher f/VT/Kg, while VT/Kg was significantly lower. Prior reports of using f/VT/Kg indicated that it did not accurately predict extubation failure. Experimental results indicate that this may still be a useful measure in certain patient populations and is a common finding in those who fail extubation readiness testing. These patients also had a statistically higher PIP, PEEP and MAP than the no device group at the time prior to extubation. For example this parameter indicates that patients within the NIV support subset did not meet the standard for adequate drive and VT on minimal settings prior to extubation. It is possible that the team decided to proceed with extubation from higher settings because these patients had underlying disease and/or required NIV support at baseline. The Cdyn values for the NIV support subset may be significantly lower, arguing that there may have been underlying lung disease. It is also possible that the decision was made to extubate these patients prior to full recovery with the intent of using NIV to support their ongoing recovery. In the pediatric population, mechanical may require higher levels of sedation than NIV ventilation. As long as there is adequate seal and the patient can protect his/her airway, non-invasive ventilation may be safe and have fewer complications than invasive mechanical ventilation.

The Cdyn value may indicate lung disease, but may also indicate that patients are not strong enough to generate the effort to overcome their diminished pulmonary compliance. This may be attributed to a variety of factors including baseline neuromuscular disease, sedative medications, myopathy or atrophy. Prolonging mechanical ventilation until parameters are fully resolved may be potentially dangerous and prolong ICU stay by exposing patients to further risk for deconditioning. Patients within this subset are an ideal group to extubate to NIV support. Cdyn may be measured by a signal that can be measured breath-to-breath and trended, which may predict the need for NIV ventilation post-extubation. Cdyn may be measured during active effort by the patient. Therefore Cdyn does not only provide insight into the lung compliance, but also if the patient is able to overcome decreasing compliance with increased effort.

The patient group requiring reintubation demonstrated significantly lower FiO₂ prior to extubation. They maintained similar end tidal carbon dioxide levels (EtCO₂) to the other groups and were on relatively low levels of ventilatory support. The reintubation subset did have a statistically significant increase in MV/Kg, which may reflect increased metabolic demand and increased production of carbon dioxide, or altered respiratory mechanics in the context of neurologic, musculoskeletal or airway abnormality. Otherwise there was no statistically significant difference in Cdyn, the extubation indices or other measured values. The lower FiO2 at time of extubation may support the fact that these patients likely did not have significant ongoing lung disease. This data reflects that reintubation in the pediatric ICU is multifactorial and not well predicted by pulmonary parameters only. Reasons for reintubation may include neuromuscular weakness, over sedation or upper airway compromise.

While there have been shown and described illustrative embodiments that include specific categories, those skilled in the art will understand than there may be other ways to predict the response of a subject undergoing respiratory therapy using a ventilator after an extubation procedure, thus the illustrative embodiment of the present invention should not be limited as such. For example, other embodiments may include different combinations of categories or different categories entirely, without deviating from the teachings herein. Furthermore, although some medical devices have been provided, the illustrative embodiment of the present invention can utilize data from any number of medical devices and may be displayed on any number of computerized devices, such as mobile phone, smartphone, computer, laptop computer, etc. Also, although the above technique has been described as being processed in a particular order, the illustrative embodiment is not necessarily limited as such since.

The foregoing description has been directed to specific embodiments. It will be apparent; however, that other variations and modifications may be made to the described embodiments, with the attainment of some or all of their advantages. For instance, it is expressly contemplated that the components and/or elements described herein can be implemented as software being stored on a tangible (non-transitory) computer-readable medium (e.g., disks/CDs/RAM/EEPROM/etc.) having program instructions executing on a computer, hardware, firmware, or a combination thereof. Accordingly this description is to be taken only by way of example and not to otherwise limit the scope of the embodiments herein. Therefore, it is the object of the appended claims to cover all such variations and modifications as come within the true spirit and scope of the embodiments herein.

What is claimed is:

1. A method for calculating patient response to an extubation procedure, the method comprising:
receiving one or more physiologic, pulmonary mechanism or oxygenation-related parameters measurements for a predetermined period of time;
retrieving a known data set;
calculating an extubation index with the physiologic or pulmonary mechanism measurements;
determining the data for mathematical multivariate modeling;
comparing the calculation with the known data set; and
producing an assessment concerning utilization of a device after extubation.

2. The method of claim 1, wherein the physiologic measurements comprise at least one of the following a measured heart rate, a measured blood pressure of the patient, a rate pressure product value, or a pulse pressure value.

3. The method of claim 1, wherein the pulmonary mechanism measurements comprise at least one of the following an oxygenation parameter, a ventilation parameter, or a pressure parameter.

4. The method of claim 1, wherein the predetermined period of time is greater than two hours.

5. The method of claim 1, wherein the predetermined period of time is less than three hours.

6. The method of claim 1, wherein at least one of a plurality of the physiologic, pulmonary mechanism measurements or oxygenation-related parameters are simultaneously received and analyzed.

7. The method of claim 1, wherein at least one of a plurality of the physiologic, pulmonary mechanism measurements or oxygenation-related parameters are continuously updated and the calculation is continuously updated.

8. The method of claim 1, wherein the known data set further comprises:
evaluating a history of measurements and outcomes to detect a potential post extubation condition; and
reporting the detected post extubation condition to a user interface.

9. The method of claim 1, wherein the one or more physiologic or pulmonary mechanism measurements are continuously updated.

10. The method of claim 1, further comprising calculating a second extubation index with the physiologic or pulmonary mechanism measurements.

11. The method of claim 10, wherein the first extubation index calculation is compared to the second extubation index to predict the utilization of a device after extubation.

12. A system for predicting a state of a subject after an extubation procedure from a mechanical ventilator, comprising:
one or more network interfaces to communicate with a network;
a processor coupled to the one or more network interfaces and configured to execute one or more processes; and
a memory coupled to store a process executable by the processor, the process when executed operable to:
receive at least one physiologic, pulmonary mechanism or oxygenation-related parameters measurements regarding the subject;
compare a known data set to the at least one physiologic, pulmonary mechanism or oxygenation-related parameters measurements; and
select a predictive device utilization assessment for a subject after the extubation procedure.

13. The system of claim 12, wherein at least one physiologic, pulmonary mechanism or oxygenation-related parameters measurements regarding the subject are continuously updated from the network.

14. The system of claim 12, wherein the predictive device utilization assessment for a subject after extubation is continuously updated.

15. The system of claim 12, wherein the predictive device utilization assessment for a subject after extubation is continuously updated after at least one physiologic, pulmonary mechanism or oxygenation-related parameter measurement is updated.

16. The system of claim 12, wherein the process when executed is further operable to: evaluation a history of subject measurement conditions and operating conditions of the ventilator to detect a potential subject condition and report the detected subject condition to the user interface.

17. The system of claim 12, wherein the process when executed is further operable to receive at least one of a dynamic compliance per KG, an oxygen saturation, a fraction of inspired oxygen, and a tidal volume.

18. The system of claim 12, wherein the process when executed is further operable to receive at least one of a dynamic compliance per KG, an oxygen saturation, a fraction of inspired oxygen, and a tidal volume or a mean airway pressure.

19. A non-transitory computer readable medium containing program instructions executed by a processor, the programming instructions comprising:
program instructions that receive one or more physiologic, pulmonary mechanism or oxygenation-related parameters measurements for a predetermined period of time;
program instructions that retrieve a known data set;
program instructions that calculate a first extubation index with the physiologic or pulmonary mechanism measurements;
program instructions that compare the calculation with the known data set; and
program instructions that produce an assessment of a device after extubation.

20. The non-transitory computer readable medium of claim 19, further comprising, program instructions calculate a second extubation index with the physiologic or pulmonary mechanism measurements.

21. The method of claim 1, further comprising calculating a third extubation index with the physiologic or pulmonary mechanism measurements.

22. The method of claim 21, wherein the first extubation index calculation is compared to the third extubation index to predict the utilization of a device after extubation.

23. The method of claim 21, wherein the second extubation index calculation is compared to the third extubation index to predict the utilization of a device after extubation.
